(19) United States
(12) Patent Application Publication
(10) Pub. No.: US 2005/0096745 A1
(43) Pub. Date: May 5, 2005

(54) Intersomatic cage for lumbar fusion by transforaminal approach and its cage carrying device

(75) Inventors: Deborah Andre, Lille (FR); Eric Leroy, Saint-Nicolas Les Arras (FR); Carlos Villanueva, Barcelone (ES); Pascal Rokegem, St. Laurent Blangy (FR)

Correspondence Address: YOUNG & THOMPSON, 745 SOUTH 23RD STREET, 2ND FLOOR, ARLINGTON, VA, 22202, US

(73) Assignee: EUROSURGICAL SA, BEAURAINS (FR)

(21) Appl. No.: 10/901,137

(22) Filed: Jul. 29, 2004

(30) Foreign Application Priority Data
Oct. 29, 2003 (FR) 03 12691

(51) Int. Cl. A61F 2/44

(52) U.S. Cl. 623/17.11

(57) ABSTRACT

The intersomatic cage includes a body with a curved profile having at each end on the one hand recesses (8, 9) provided over all the height of the cage, and on the other hand in a direction perpendicular to the direction of the recesses (8, 9) a horizontal notch (14) opening into each recess (8, 9) and permitting temporarily connecting the intersomatic cage to a rod (19) of a cage carrying device (15) ensuring freedom of progressive angular rotation of the cage (1) as a function of its position of introduction between the vertebral bodies (26).

The present invention relates to an intersomatic cage permitting the fusion between two vertebral bodies of a vertebral column and to its cage carrying device adapted for its introduction between the vertebral bodies.

The intersomatic cage according to the present invention is introduced between the vertebral bodies by a transforaminal approach permitting the emplacement of a single cage to carry out lumbar fusion of the vertebral bodies.

The intersomatic cage for the fusion of vertebral bodies by transforaminal approach according to the present invention comprises a body with a curved profile having at each end on the one hand recesses provided over all the height of said cage and on the other hand in, a direction perpendicular to that of the recesses, a horizontal notch opening into each recess and permitting correcting temporarily the intersomatic cage to a rod of a cage carrying device ensuring progressive angular freedom of rotation of the cage as a function of its position of introduction between the vertebral bodies.

The intersomatic cage according to the present invention comprises over all its height two internal seats separated by a central partition connecting the anterior and posterior walls to each other.

The intersomatic cage according to the present invention comprises recesses which are separated respectively from the adjacent internal seat by a vertical partition.

The intersomatic cage according to the present invention comprises along its radius of curvature grooves provided on the upper and lower surfaces of each partition.

The intersomatic cage according to the present invention comprises two radiographic markers disposed in a vertical direction across the thickness of the anterior wall and at the level of the partitions and a radiographic marker disposed in a horizontal direction connecting the anterior and posterior walls at the level of the central partition.

The intersomatic cage according to the present invention comprises a posterior wall of smaller radius which is pierced by vascularization holes opening respectively into the internal seats.

The cage carrying device for the introduction of an intersomatic cage according to the present invention is constituted by an ergonometric handle comprising lateral flats permitting marking the position of a attachment lug located at the free end of a rod comprising at a certain distance from the handle drive means permitting moving in translation a blocking tube along said rod either to approach or to move away from the attachment lug.

The cage carrying device according to the present invention comprises drive means which are constituted by a wheel coacting by means of, on the one hand, a screw-threaded bore, with a screw-threaded portion of the rod, and on the other hand, with a seat located in prolongation of the screw-threaded bore with a head secured to the blocking tube.

The cage carrying device according to the present invention comprises at the free end of the blocking tube and at the level of the attachment lug, a profile complementary to the external profile of the ends of the intersomatic cage and more particularly complementary to the one located about notches so as to be able to match perfectly this latter during locking of said cage on said cage carrying device.

The cage carrying device according to the present invention comprises a wheel whose seat is provided with predetermined dimensions to coact with the head of the tube 25 such that said wheel can turn in rotation on the screw-threaded portion of the rod without causing rotation of said tube.

The cage carrying device according to the present invention comprises a blocking tube which is mounted slidably and without the possibility of turning about the rod so as to be able to function in the direction of rotation of the wheel either to approach or move away from the attachment lug.

The cage carrying device according to the invention comprises a rod whose attachment lug has a cylindrical profile disposed in a direction perpendicular to the principal axis of said rod.

The description which follows with respect to the accompanying drawings, given by way of non-limiting examples, permits better understanding of the invention, its characteristics and advantages which it is adapted to provide:

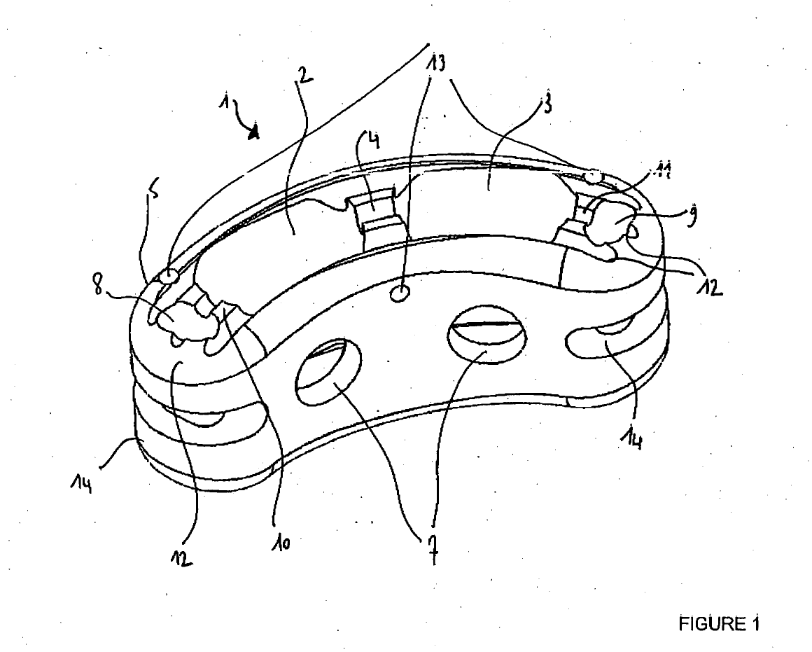

FIG. 1 is a perspective view showing the intersomatic cage according to the present invention.

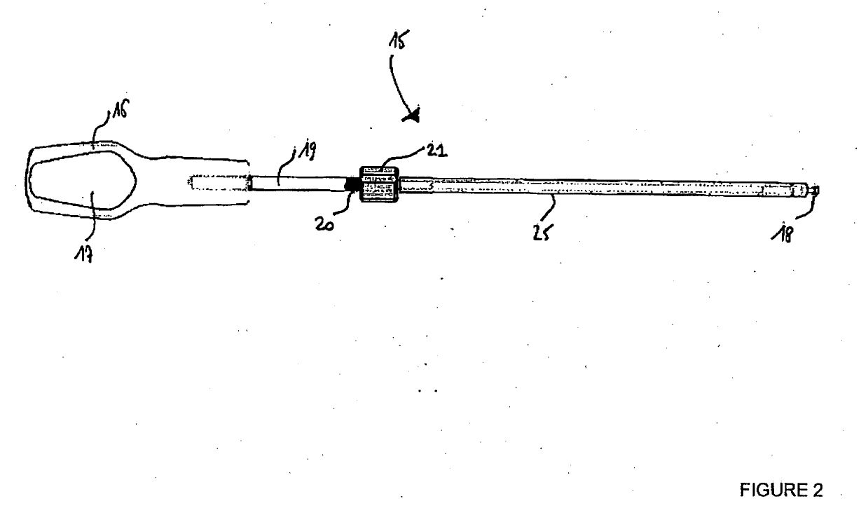

FIG. 2 is a view showing the cage carrying device permitting the introduction of the intersomatic cage between the vertebral bodies of a vertebral column.

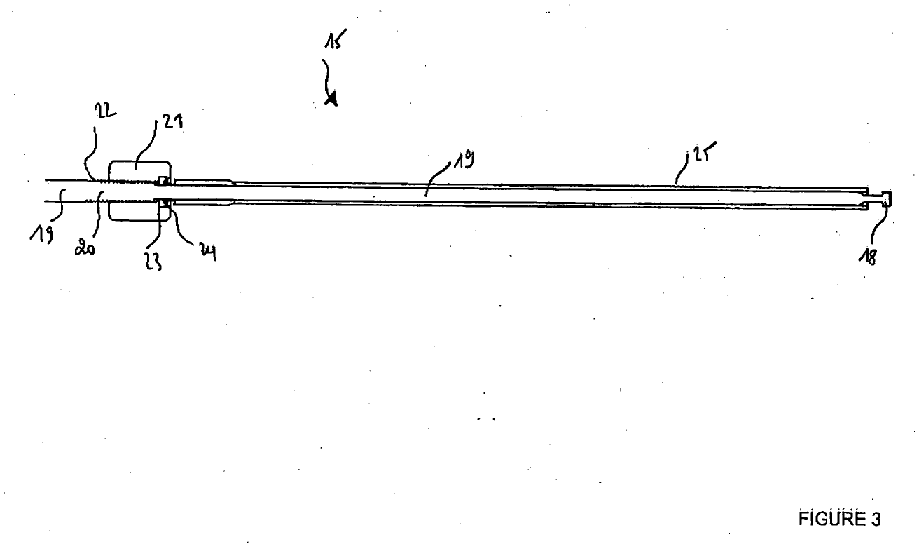

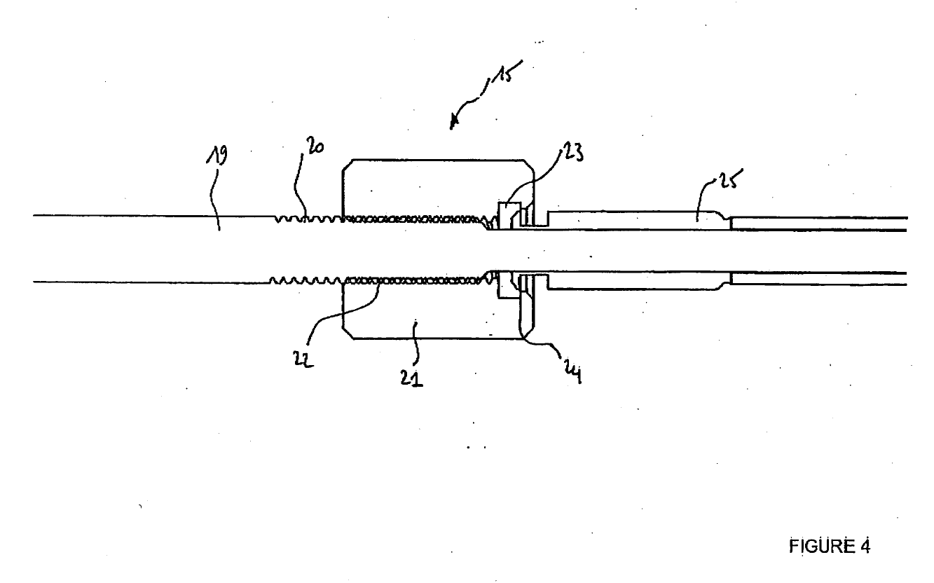

FIGS. 3 and 4 are detailed views showing the cage carrying device according to the present invention.

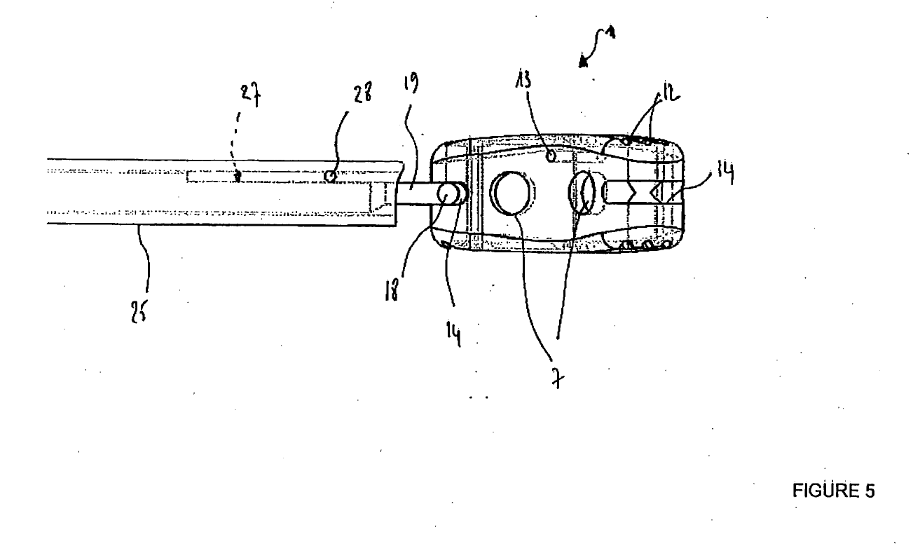

FIGS. 5 to 7 are views showing the securement of the intersomatic cage on its cage carrying device before its introduction into the vertebral bodies of a vertebral column.

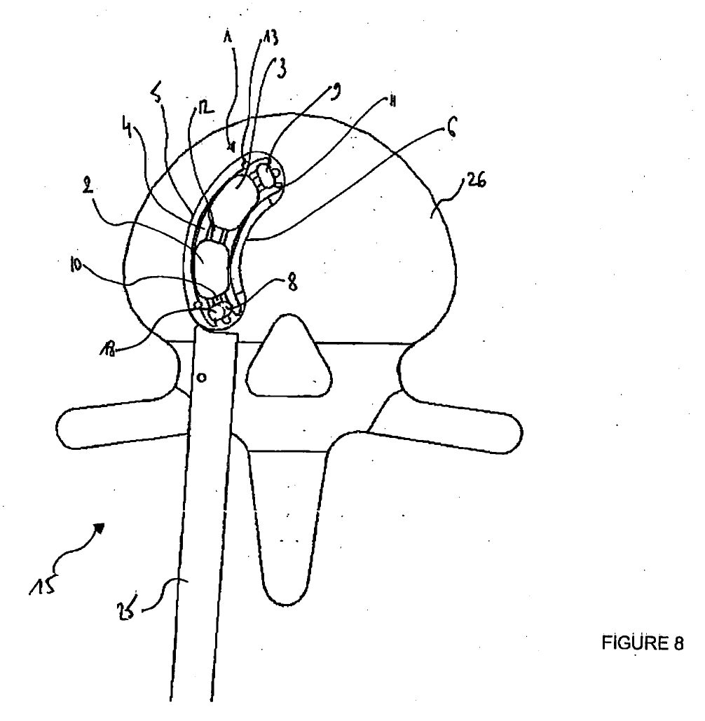

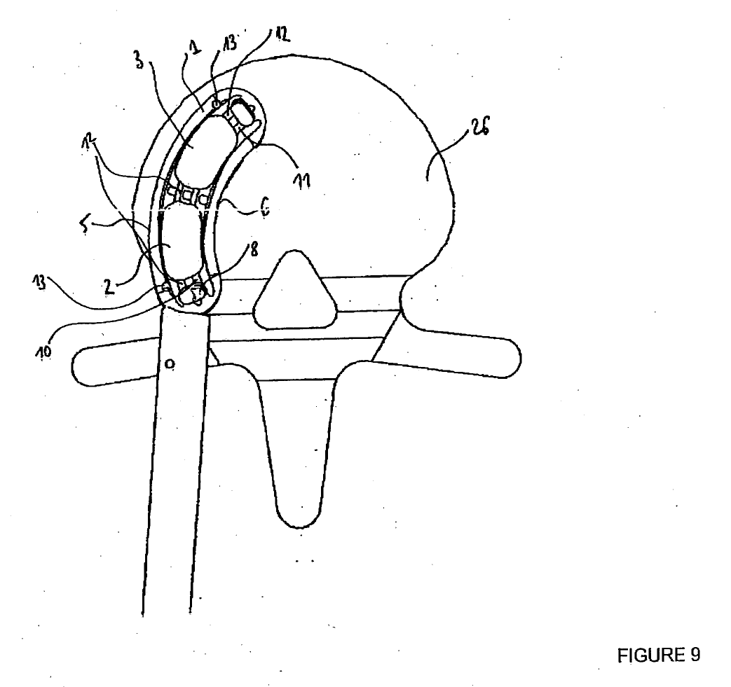

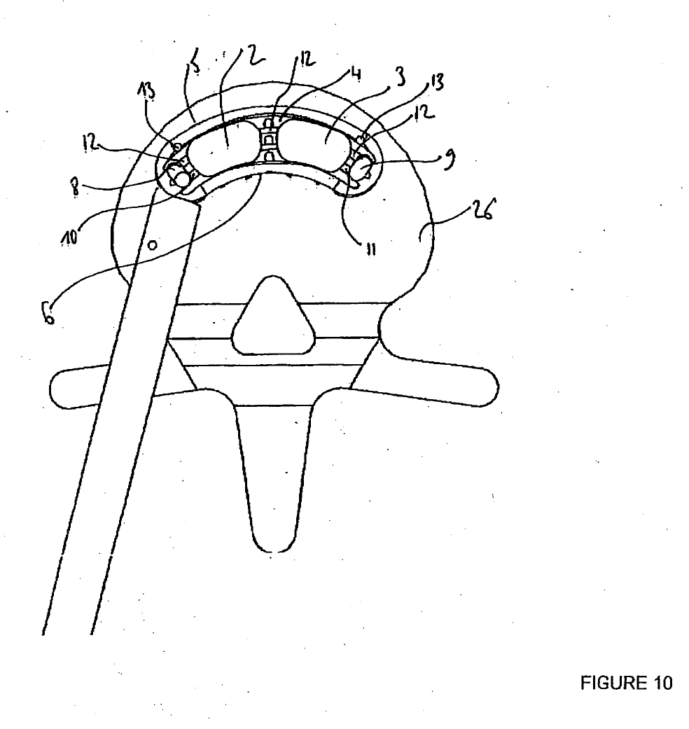

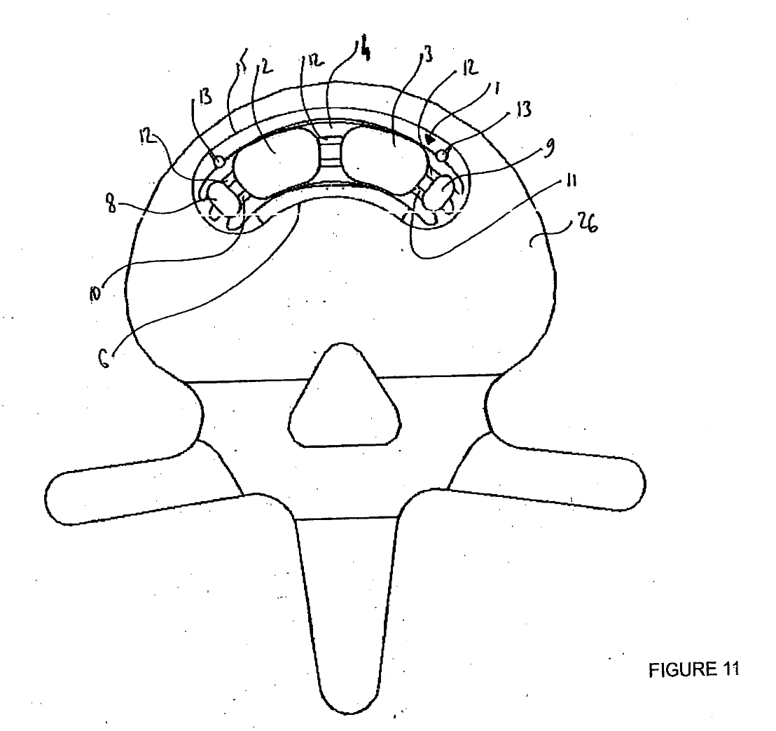

FIGS. 8 to 11 are views showing the emplacement of the intersomatic cage between the vertebral bodies of a vertebral column by means of the cage carrying device.

There is shown in FIG. 1 an intersomatic cage 1 having an external profile that is curved or of kidney shape and of which all the edges are smoothed principally at the two ends.

The intersomatic cage 1 is asymmetric in the horizontal plane and bisymmetric in the vertical plane, permitting carrying out an approach or an introduction either from the left or from the right of the vertebral column without having to change the cage. Thus, the upper radius of curvature is smaller than the lower radius of curvature. The horizontal marker 13 is located adjacent the upper edge.

The intersomatic cage 1 comprises over all its height two internal seats 2 and 3 separated by a central partition 4 connecting the anterior wall 5 and the posterior wall 6 to each other.

The posterior wall 6 of smaller radius is pierced by vascularization holes 7 opening respectively into the internal seats 2 and 3 of the intersomatic cage 1.

The intersomatic cage 1 comprises at each end and over all its height recesses 8 and 9 separated respectively from the adjacent internal seat 2, 3 by a vertical partition 10, 11.

The upper and lower surfaces of the intersomatic cage 1 comprise at each end and on each partition 4, 10 and 11, grooves 12 disposed along its radius of curvature.

The intersomatic cage 1 comprises radiographic markers 13 made of tantalum wires, which are positioned in the following manner:
two markers 13 disposed in a vertical direction over the thickness of the anterior wall 5 and at the level of the partitions 10 and 11,
a marker 13 disposed in a horizontal direction connecting the anterior wall 5 and posterior wall 6 at the level of the central partition 4.

The intersomatic cage 1 comprises at each end a horizontal notch 14 opening perpendicularly into each recess 8, 9, permitting providing the connection of said cage with a cage carrying device 15.

There is shown in FIGS. 2 to 4 a cage carrying device 15 permitting the introduction of the intersomatic cage 1 between the vertebral bodies of a vertebral column.

The cage carrying device 15 is constituted by an ergonometric handle 16 comprising lateral flats 17 permitting marking the position of the attachment lug 18 located at the opposite end from the handle.

The ergonometric handle 16 is secured to a rod 18 comprising at its free end the attachment lug 18 having a cylindrical profile disposed in a direction perpendicular to the principal axis of the rod 19.

The rod 19 comprises, at a certain distance from the handle 16, a screw-threaded portion 20 adapted to coact with a screw-threaded wheel 21. This latter comprises, in the prolongation of its screw-threaded bore 22, a seat 23 of larger diameter so as to be able to receive the head 24 of a blocking tube 25.

The seat 23 is provided with predetermined dimensions to coact with the head 24 of the tube 25 such that the wheel 21 can turn in rotation on the screw-threaded portion 22 of the rod 19, without driving said tube 25 in rotation. This blockage in rotation is obtained by the co-action of a flap 27 provided on the rod 19 and a pin 28 in the tube 25.

The blocking tube 25 is mounted slidably and without the possibility of turning about the rod 19 so as to be able, as a function of the direction of rotation of the wheel 21, either to approach or to move away from the attachment lug 18.

Thus, the wheel 21 permits, when it is driven in rotation on the screw-threaded portion 20 of the rod 19, moving in translation the blocking tube 25 along said rod either to move toward or away from the attachment lug 18.

The free end of the blocking tube 25 comprises, at the level of the attachment lug 18, a profile complementary to the external profile of the ends of the intersomatic cage 1 and more particularly the external profile located about the notches 14, so as to be able to match perfectly this latter during locking of said cage on the cage turning device 15.

There is shown in FIGS. 5 to 8 the different steps of emplacing the intersomatic cage 1 on the cage carrying device 15 before its introduction between the vertebral bodies of a vertebral column.

To lock the intersomatic cage 1 on the cage carrying device 15, there is first introduced the attachment lug 18 into the corresponding recess 8, 9 through the notch or slot 14 (FIG. 5).

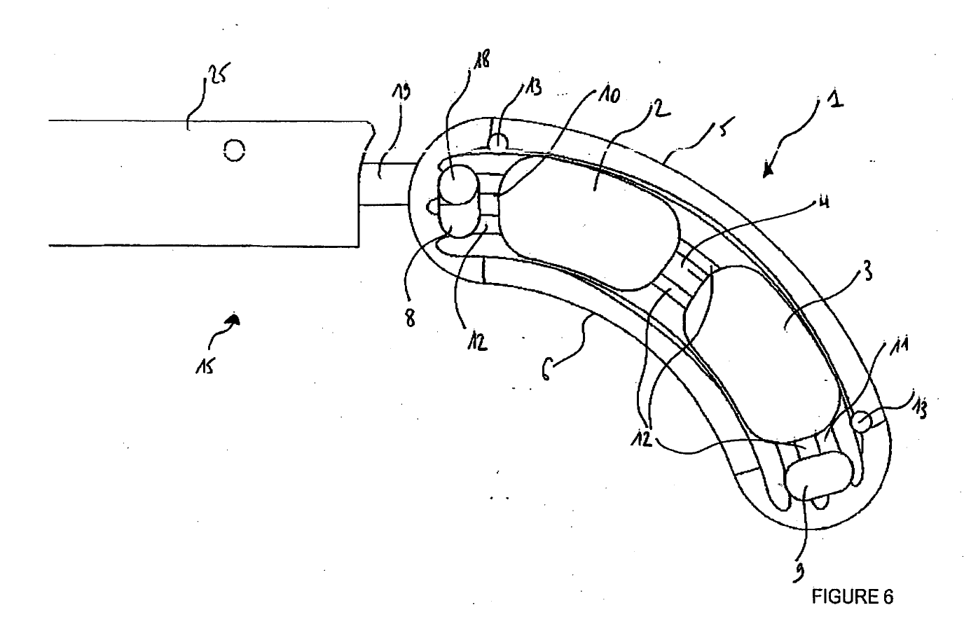

There is then performed a rotation of 90 degrees between the intersomatic cage 1 and the cage carrying device 15, so as to block the attachment lug 18 within the corresponding recess 8, 9 (FIG. 6).

The blocking tube 25 is moved in translation by means of the wheel 21 until the external wall of the intersomatic cage 1 comes into engagement against the end of the blocking tube 25.

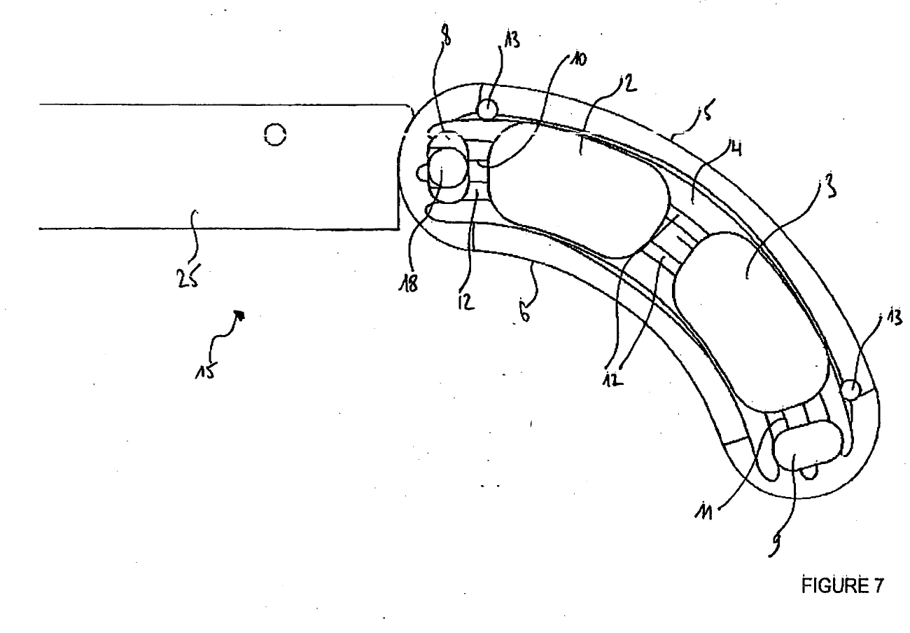

Thus, the external wall of the intersomatic cage 1 will be gripped between the end with a complementary profile of the tube 25 and the attachment lug 18, permitting temporarily securing the intersomatic cage 1 to the cage carrying device 15 (FIG. 7).

There is shown in FIGS. 8 to 11 the introduction of the intersomatic cage 1 fixed temporarily to the end of the cage carrying device 15, between the vertebral bodies 26 of a vertebral column.

One of the constraints is to limit as much as possible the size of the opening provided between the vertebral bodies 26 and through which the intersomatic cage 1 and the different instruments will pass.

To do this, the intersomatic cage 1 is located in prolongation of the cage carrying device 15, because it is in this position that it occupies the least space.

The cage carrying device 15 then ensures the progressive rotation of the intersomatic cage 1 between the vertebral bodies 26 until there is an angle of about 90 degrees between said cage and the cage carrying device 15 and thus the cage is in its final position relative to the vertebral bodies.

The progressive rotation of the intersomatic cage 1 relative to the cage carrying device 15 is effected by the pressure exerted by the surgeon on the handle 16 of the cage carrying device 15, while the intersomatic cage 1 is gripped between the vertebral bodies 26.

The surgeon proceeds to the different positionings of the cage carrying device 15 relative to the intersomatic cage 1 gripped between the vertebral bodies 26, by successively moving the tube 25 along the rod 19 until said cage will be in its final position.

To release completely the intersomatic cage 1 from the cage carrying device 15, it is necessary to turn the wheel 21 so as to distance the tube 25 from said cage and to move the attachment lug 18 out of the corresponding recess 8, 9 by rotative manipulation of the handle 16 by 90 degrees.

These different steps of positioning the cage carrying device 15 relative to the intersomatic cage 1 are facilitated by the flats 17 which permit the surgeon to visualize the position of the attachment lug 18.

To this end, the latter is located in the same plane as that in which the flats 17 are located.

It should further be understood that the above description was given only by way of example and that it in no way limits the scope of the invention, which will not be exceeded by replacing the described details of execution by any other equivalent.

1. Intersomatic cage for the fusion of vertebral bodies (26) by transforaminal approach, characterized in that it comprises a body with a curved profile having at each end on the one hand recesses (8, 9) provided over all the height of said cage and on the other hand in a direction perpendicular to that of the recesses (8, 9) a horizontal notch (14) opening into each recess (8, 9) and permitting temporarily connecting the intersomatic cage to a rod (19) of a cage carrying device (15) ensuring progressive freedom of angular rotation of the cage (1) as a function of its position of introduction between the vertebral bodies (26).

2. Intersomatic cage according to claim 1, characterized in that it comprises over all its height two internal seats (2, 3) separated by a central partition (4) connecting the anterior wall (5) and the posterior wall (6) to each other.

3. Intersomatic cage according to claim 2, characterized in that the recesses (8, 9) are separated respectively from the adjacent internal seat (2, 3) by a vertical partition (10, 11).

4. Intersomatic cage according to claim 3, characterized in that it comprises, along its radius of curvature, grooves (12) provided on the upper and lower surfaces of each partition (4, 10 and 11).

5. Intersomatic cage according to claim 2, characterized in that it comprises two radiographic markers (13) disposed in a vertical direction over the thickness of the anterior wall (5) and at the level of the partitions (10, 11) and the marker (13) disposed in a horizontal direction connecting the anterior wall (5) and posterior wall (6) at the level of the central partition (4).

6. Intersomatic cage according to claim 2, characterized in that the posterior wall (6) of smaller thickness is pierced by vascularization holes (7) opening respectively into the internal seats (2, 3).

7. Cage carrying device for the introduction of an intersomatic cage (1) according to claim 1, characterized in that it is constituted by an ergonometric handle (16) comprising lateral flats (17) permitting marking the position of an attachment lug (18) located at the free end of a rod (19) comprising at a certain distance from the handle (16) drive means (21) permitting moving in translation a blocking tube (25) along said rod (19) either to approach or to move away from the attachment lug (18).

8. Cage carrying device for the introduction of an intersomatic cage (1) according to claim 7, characterized in that the drive means are constituted by a wheel (21) coacting by means on the one hand of a screw-threaded bore (22) with a screw-threaded portion (20) of the rod (19), and on the other hand with a seat (23) located in prolongation of the screw-threaded bore (22) with a head (24) secured to the blocking tube (25).

9. Cage carrying device for the introduction of an intersomatic cage (1) according to claim 7, characterized in that the free end of the blocking tube (25) comprises at the level of the attachment lug (18) a profile complementary to the external profile of the ends of the intersomatic cage (1) and more particularly complementary to the one located about notches (14) so as to be able to match perfectly this latter during locking of said cage on the cage carrying device (15).

10. Cage carrying device for the introduction of an intersomatic cage (1) according to claim 8, characterized in that the seat (23) is provided with predetermined dimensions to coact with the head (24) of the tube (25) such that the wheel (21) can turn in rotation on the screw-threaded portion (22) of the rod (19) without causing rotation of said tube (25).

11. Cage carrying device for the introduction of an intersomatic cage (1) according to claim 8, characterized in that the blocking tube (25) is mounted slidably and without the possibility of turning about the rod (19) so as to be able, as a function of the direction of rotation of the wheel (21), either to approach or to move away from the attachment lug (18).

12. Cage carrying device for the introduction of an intersomatic cage (1) according to claim 1, characterized in that the attachment lug (18) has a cylindrical profile disposed in a direction perpendicular to the principal axis of the rod (19).
